(19) United States
(12) Patent Application Publication
(10) Pub. No.: US 2005/0113727 A1
(43) Pub. Date: May 26, 2005

(54) Post-surgical arm protector

(76) Inventor: Angie Page, Cody, WY (US)

Correspondence Address: KLARQUIST SPARKMAN, LLP, 121 SW SALMON STREET, SUITE 1600, PORTLAND, OR, 97204, US

(21) Appl. No.: 10/967,077

(22) Filed: Oct. 14, 2004

Related U.S. Application Data
(60) Provisional application No. 60/512,026, filed on Oct. 17, 2003.

(51) Int. Cl. A61F 5/00

(52) U.S. Cl. 602/4

(57) ABSTRACT

The present application concerns a trauma or post surgical device comprising a universally recognized warning concerning the trauma or surgical site. One embodiment concerns a sling having the universally recognized warning, and may further include a garment attached to the sling. A method also is described, one embodiment of which concerns coupling a warning garment to a sling.

### CROSS REFERENCE TO RELATED APPLICATION

This application claims the benefit of the earlier filing date of U.S. Provisional Application No. 60/512,026, filed on Oct. 17, 2003. The entire disclosure of provisional application No. 60/512,026 is considered to be part of the disclosure of the accompanying application and is incorporated herein by reference.

### BACKGROUND

Current orthopedic practice for patients with shoulder and/or elbow surgeries, fractures and dislocations, provides a post-surgical/post-reduction arm sling that supports the shoulder, forearm and wrist to reduce mobility and provide time for healing. These slings are typically constructed of a fabric pouch supporting the forearm and are equipped with adjustable straps and/or buckles. However, they do not provide the patient with any coverage, protection or warning to others of the post-surgical/trauma site, which is often tender and painful to the touch. Therefore, there is a clear need for this in the industry.

### BRIEF SUMMARY

A sling, or an accessory garment for attachment to currently known slings, particularly arm slings, is described. Disclosed embodiments of the invention provide the patient with protection to the surgical/trauma area by reducing or altogether eliminating the possibility of erroneous contact from other individuals, which causes additional, unnecessary pain and/or possible post-surgical trauma.

A first embodiment comprises a garment for attachment to known slings. The garment can be constructed from any suitable durable, yet preferably washable, fabric. The garment also preferably, but necessarily, is made from a brightly colored fabric, such as yellow, orange or red, in order to draw attention.

The garment fits individuals of different sizes. For example, the garment can be attached to the arm sling at specific intervals on the underneath side of the garment, as well as the top front and top back of the sling. In order to attach the garment to existing slings, any type of fastener may be used as long as it provides minimal difficulty for the patient to apply. Such fastening devices include Velcro, snaps, hooks, buttons, and combinations thereof.

Additionally, the garment will include a warning, such as an imprinted warning, to others not to touch injured or post surgical sites. For example, the garment may display a warning, such as the universal circle with slash symbol, surrounding and intersecting a word, such as “TOUCH” (indicating “do not touch”), or any other appropriate term, English or foreign, and/or further noting that the area is a “POST-SURGICAL SITE” or other suitable wording. This is placed on, such as imprinting, on both the front and back of the garment. Additionally, if desired, a type of padding may be applied to the underneath side of the shoulder area for extra comfort to the patient.

The garment is especially useful when worn while the patient is out in public when the greatest likelihood of contact may occur. This offers the patient a form of nonverbal communication to the general population to approach them with extra caution and to be cognizant that the area has undergone recent trauma.

### BRIEF DESCRIPTION

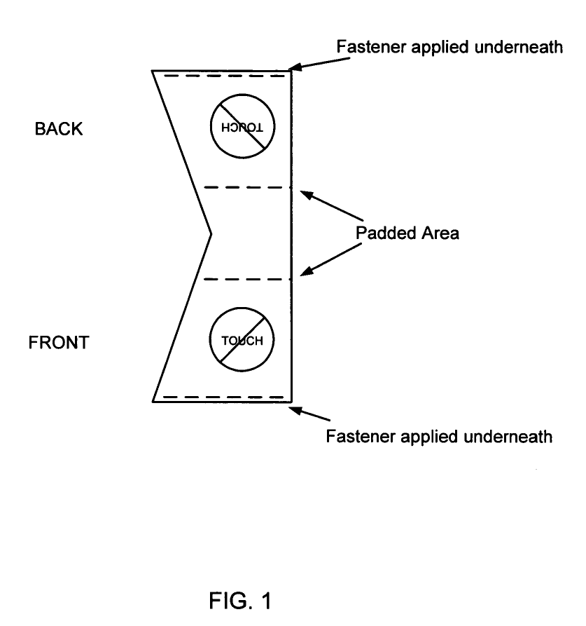

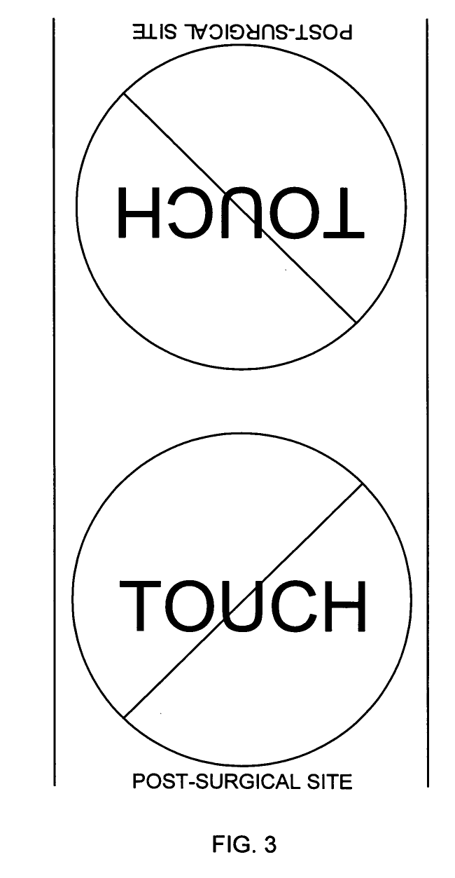

FIGS. 1 and 3 illustrate embodiments of a garment according to the present invention.

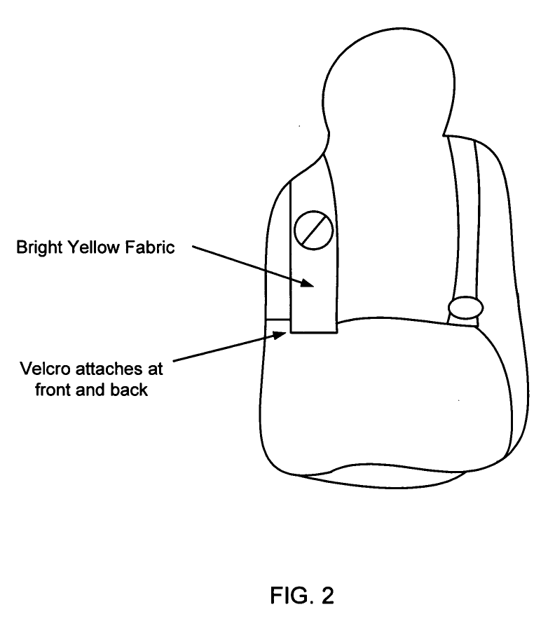

FIG. 2 illustrates use of a disclosed garment coupled to an arm sling.

The present invention has been described with respect to particular embodiments. Persons of ordinary skill in the art will recognize that the invention is not limited in scope solely to those features disclosed.

1. A trauma or post surgical device, comprising a universally recognized warning concerning the trauma or surgical site.

2. The device according to claim 1 comprising a sling having the universally recognized warning.

3. The device according to claim 2 comprising a garment attached to the sling.

4. The device according to claim 3 coupled to the sling with a fastener.

5. The device according to claim 4 made from a brightly colored fabric.

6. A method, comprising:
providing a garment that provides a warning concerning an injury or post-surgical site; and
coupling the garment to a sling.
